(19) United States
(12) Patent Application Publication
(10) Pub. No.: US 2023/0390436 A1
(43) Pub. Date: Dec. 7, 2023

(54) ULTRAVIOLET LIGHT IRRADIATION SYSTEM AND ULTRAVIOLET LIGHT IRRADIATION METHOD

(71) Applicant: NIPPON TELEGRAPH AND TELEPHONE CORPORATION, Tokyo (JP)

(72) Inventors: Tomohiro TANIGUCHI, Musashino-shi, Tokyo (JP); Ayako IWAKI, Musashino-shi, Tokyo (JP); Kazuhide NAKAJIMA, Musashino-shi, Tokyo (JP); Nobutomo HANZAWA, Musashino-shi, Tokyo (JP); Takashi MATSUI, Musashino-shi, Tokyo (JP); Yuto SAGAE, Musashino-shi, Tokyo (JP); Chisato FUKAI, Musashino-shi, Tokyo (JP)

(73) Assignee: NIPPON TELEGRAPH AND TELEPHONE CORPORATION, Tokyo (JP)

(21) Appl. No.: 18/032,514

(22) PCT Filed: Oct. 21, 2020
(86) PCT No.: PCT/JP2020/039582

§ 371 Date: Apr. 18, 2023

(51) Int. Cl. A61L 2/10

(52) U.S. Cl. CPC A61L 2/10; A61L 2202/11

(57) ABSTRACT

An object of the present invention is to provide an ultraviolet light irradiation system and an ultraviolet light irradiation method which are not limited in an ultraviolet light irradiation region and can be introduced economically. The present ultraviolet light irradiation system is a system configured to connect a single ultraviolet light source part and an irradiation part installed in the vicinity of an object place to be sterilized or the like by the optical cable formed by bundling a plurality of optical fibers (the single-core or the multi-core) or the multi-core optical fiber. Since the optical fiber is very thin and does not require power supply for transmitting the ultraviolet rays, even in a fine place where a person or a robot cannot enter, the ultraviolet light can be irradiated by laying the optical fiber or the optical cable.

### TECHNICAL FIELD

The present disclosure relates to an ultraviolet light irradiation system in which a light source and an ultraviolet light irradiation part are separated from each other, and an ultraviolet light irradiation method thereof.

### BACKGROUND ART

For the purpose of preventing infectious diseases, there is an increasing demand for systems for sterilization and inactivation of viruses using the ultraviolet light. Systems have three major categories of products. In the following description, sterilization and virus inactivation are described as “sterilization or the like”.

(1) Mobile Sterilization Robot (for example, Refer to NPL 1)

A mobile sterilization robot is an autonomous mobile robot for radiating the ultraviolet light. The robot radiates the ultraviolet rays while moving in a room in a building such as a hospital room, thereby automatically achieving the sterilization or the like over a wide range without human intervention.

(2) Stationary Air Purifier (for example, Refer to NPL 2)

A stationary air purifier is a device that is installed on a ceiling or in a predetermined place in a room and performs the sterilization or the like while circulating the air in the room. Since the device does not directly radiate the ultraviolet light and does not affect the human body, highly safe sterilization or the like can be performed.

(3) Portable Sterilization Device (for example, Refer to NPL 3)

A portable sterilization device is a portable type device mounted with an ultraviolet light source. A user can use the device in various places by carrying the device to an area where is an object of the sterilization or the like and irradiating the object of the sterilization or the like with the ultraviolet light.

### Citation List

[Non Patent Literature]

[NPL 1] The website of Kantum Ushikata Co., Ltd. (https://www.kantum.co.jp/product/sakkin_robot/sakkinn_robot/UVD_robot)

[NPL 2] The website of IWASAKI ELECTRIC CO., Ltd. (https://www.iwasaki.co.jp/optics/sterilization/air/air03.html)

[NPL 3] The website of Funakoshi Co., Ltd. (https://www.funakoshi.co.jp/contents/68182)

### SUMMARY OF INVENTION

### Technical Problem

However, the above mentioned systems of NPLs have the following problems.

In the system of NPL 1, the object place of the sterilization or the like is limited to a place where the robot can move and enter, and it is difficult to sterilize or the like a fine place, a deep place, or the like.

Since the system of NPL 2 is a method for performing the sterilization or the like by circulating indoor air, it is difficult to directly irradiate a place where the sterilization or the like is desired with the ultraviolet light.

In the system of NPL 3, for example, the ultraviolet light cannot be irradiated to an area where a narrow pipeline is not put or a person cannot enter.

In other words, the above-described systems of NPLs have a first problem that it is difficult to perform the sterilization or the like to a desired place.

Further, in the system of NPL 1, since the sterilization robot radiates high-output ultraviolet light, the device is large-scale and expensive, and there is a second problem that it is difficult to realize an economical system.

Thus, in order to solve the above problems, an object of the present invention is to provide an ultraviolet light irradiation system and an ultraviolet light irradiation method have no limitation in an ultraviolet light irradiation region and can be economically introduced.

### Solution to Problem

In order to achieve the above object, the ultraviolet light irradiation system according to the present invention propagates an ultraviolet light from a light source part by a spacial multiplex transmission scheme.

Specifically, the ultraviolet light irradiation system according to the present invention includes
an ultraviolet light source part for outputting the ultraviolet light,
an optical transmission part for propagating the ultraviolet light by the spatial multiplex transmission scheme, and
an irradiation part for irradiating a desired place with the ultraviolet light propagated by the optical transmission part.

The ultraviolet light irradiation method is characterized in that when the ultraviolet light outputted from the ultraviolet light source part is irradiated from the irradiation part to the desired place, the ultraviolet light is propagated by a spatial multiplex transmission scheme.

As an example of realizing the spatial multiplex transmission scheme, it is characterized in that the optical transmission part is an optical cable in which a plurality of single-core optical fibers is bundled, one multi-core optical fiber, or an optical cable in which a plurality of multi-core optical fibers is bundled.

For example, the single-core optical fiber is any one of a solid core optical fiber, a hole-assisted optical fiber, a hole-structured optical fiber, a hollow core optical fiber, and a coupled core type optical fiber. In addition, the multi-core optical fiber is any one of a solid core type multi-core optical fiber, a hole-assisted type multi-core optical fiber, a hole-structured type multi-core optical fiber, a hollow core type multi-core optical fiber, and a coupled core type multi-core optical fiber.

It is characterized in that the plurality of irradiation parts are provided, and an optical distribution part for distributing the ultraviolet light propagated by the optical transmission part to the respective irradiation parts is further provided. For example, the irradiation part and the optical distribution part are connected by ant one of the solid core optical fiber, the hole-assisted optical fiber, the hole-structured optical fiber, the hollow core optical fiber, the coupled core type optical fiber, the solid core type multi-core optical fiber, the hole-assisted type multi-core optical fiber, the hole-structured type multi-core optical fiber, the hollow core type multi-core optical fiber, and the coupled core type multi-core optical fiber.

The present ultraviolet light irradiation system is a system configured to connect a single ultraviolet light source part and an irradiation part installed in the vicinity of an object place to be sterilized or the like by the optical cable formed by bundling a plurality of optical fibers (the single-core or the multi-core) or the multi-core optical fiber. Since the optical fiber is very thin and does not require power supply for transmitting the ultraviolet rays, even in a fine place where a person or a robot cannot enter, the ultraviolet light can be irradiated by laying the optical fiber or the optical cable. Accordingly, the present ultraviolet light irradiation system can resolve the first problem described above.

In addition, the present ultraviolet light irradiation system transmits the ultraviolet light by the spatial multiplex transmission scheme, thereby, sufficient power can be transmitted without being restricted by the limit of output power of the single light source or transmission power of the single optical fiber or core. Therefore, the present ultraviolet light irradiation scheme has an advantage that it is possible to sterilize or the like in a wide range and in a short time.

Further, the ultraviolet light irradiation system may be configured so that the single ultraviolet light source part and the plurality of irradiation parts respectively installed in the vicinity of the plurality of object places are connected by the optical cable or the multi-core optical fiber vis the optical distribution part. By this configuration, the single ultraviolet light source part can be shared in work such as sterilization or the like to the plurality of object places, and the whole system can be made an economical configuration. Therefore, the present ultraviolet light irradiation system can also resolve the second problem.

As described above, the present invention can solve the first problem and the second problem, and can provide the ultraviolet light irradiation system and the ultraviolet light irradiation method which have no limitation on the irradiation region of the ultraviolet light, and can be introduced economically.

It should be noted that the inventions described above can be combined where possible.

### Advantageous Effects of Invention

The present invention can provide an ultraviolet light irradiation system and an ultraviolet light irradiation method which have no limitation on an ultraviolet light irradiation region and can be introduced economically.

### BRIEF DESCRIPTION OF DRAWINGS

FIG. 1 is a diagram for describing an ultraviolet light irradiation system according to the present invention.

FIG. 2 is a diagram for describing an optical transmission part of the ultraviolet light irradiation system according to the present invention.

FIG. 2 is a diagram for describing an optical transmission part of the ultraviolet light irradiation system according to the present invention.

FIG. 2 is a diagram for describing an optical transmission part of the ultraviolet light irradiation system according to the present invention.

FIG. 5 is a diagram for describing a cross section of an optical fiber.

FIG. 6 is a diagram for describing a light source of the ultraviolet light irradiation system according to the present invention.

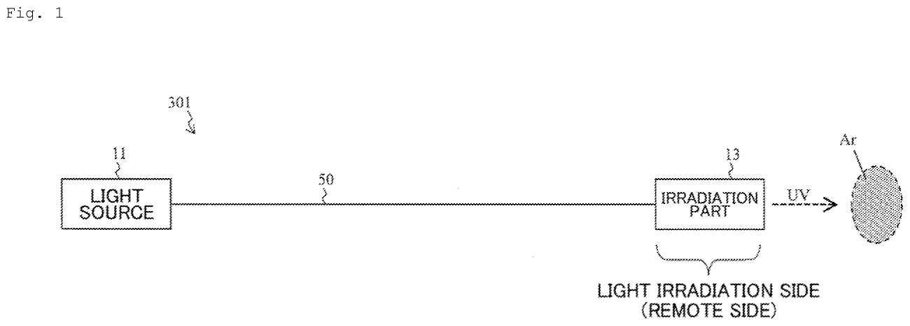

FIG. 1 is a diagram for describing an ultraviolet light irradiation system according to the present invention.

FIG. 1 is a diagram for describing an ultraviolet light irradiation system according to the present invention.

FIG. 9 is a diagram for describing an ultraviolet light irradiation method according to the present invention.

### DESCRIPTION OF EMBODIMENTS

Embodiments of the present invention will be described with reference to accompanying drawings. The embodiments described below are examples of the present invention and the present invention is not limited to the following embodiments. Note that constituent elements with the same reference signs in the present description and the drawings are identical to each other.

### Embodiment 1

FIG. 1 is a diagram for describing an ultraviolet light irradiation system 301 of the present embodiment. The ultraviolet light irradiation system 301 includes
an ultraviolet light source part 11 for outputting the ultraviolet light,
an optical transmission part 50 for dividing the ultraviolet light and propagating the divided ultraviolet light by a spatial multiplex transmission scheme,
an irradiation part 13 for irradiating a desired place with the ultraviolet light.

The irradiation part 13 irradiates a desired object place Ar of sterilization or the like with the ultraviolet light propagated by the optical transmission part 50. The irradiation part 13 is configured of an optical system such as a lens designed for the wavelength of ultraviolet region.

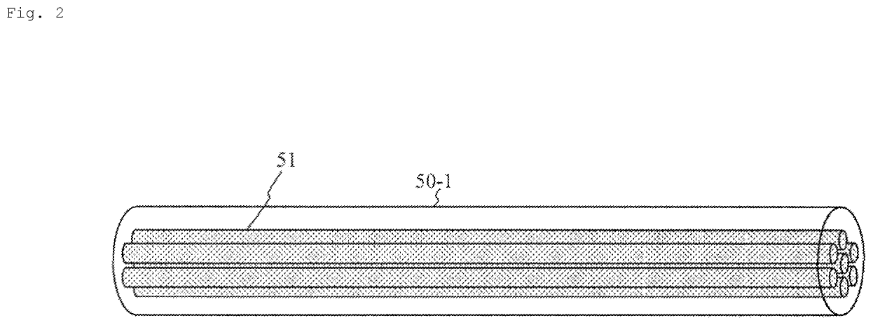

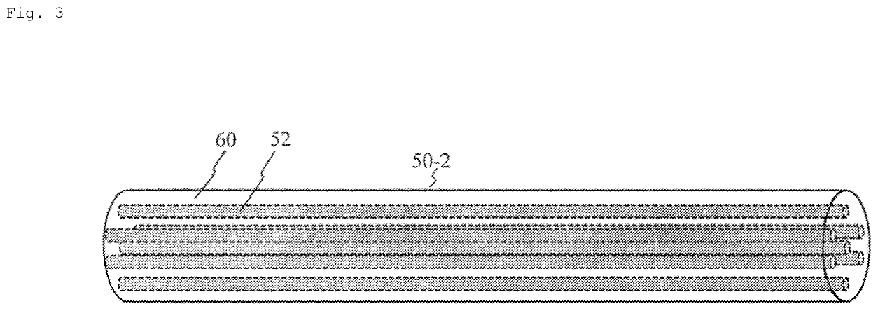

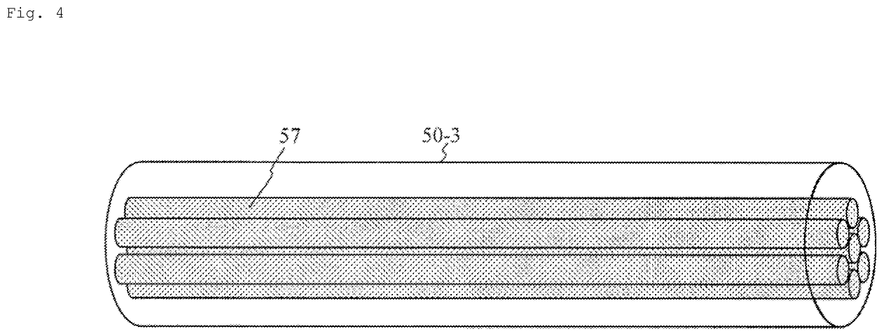

FIG. 2 is a diagram for describing a form of the optical transmission part 50. That is, the optical transmission part 50 is an optical cable 50-1 formed by bundling a plurality of single-core optical fibers 51.

FIG. 3 is a diagram for describing a form of the optical transmission part 50. That is, the optical transmission part 50 is a single multi-core optical fiber 50-2 having a plurality of cores 52.

FIG. 4 is a diagram for describing a form of the optical transmission part 50. That is, the optical transmission part 50 is an optical cable 50-3 formed by bundling a plurality of multi-core optical fibers 57.

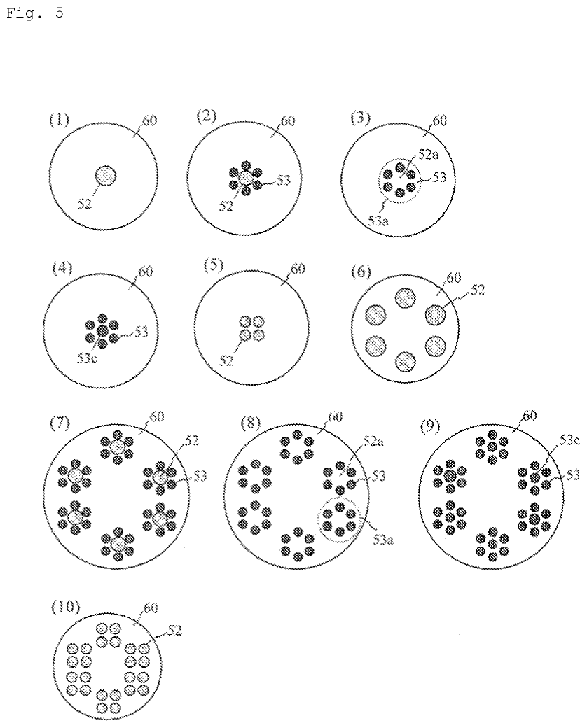

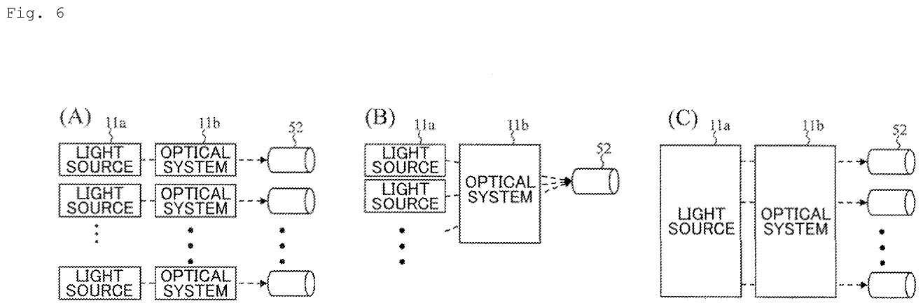

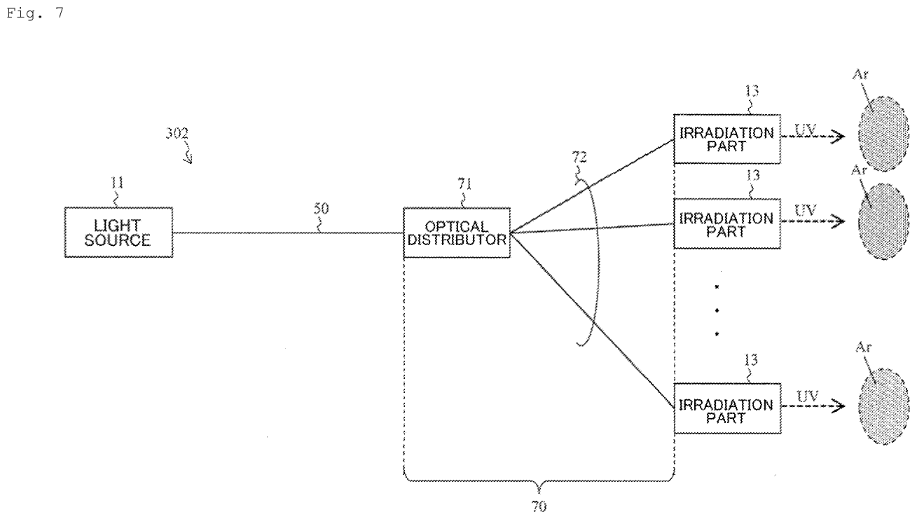

FIG. 5 is a diagram for describing a cross section of the optical fiber. As the single-core optical fiber 51, an optical fiber having a cross-sectional structure as shown in FIGS. 5 (1) to (5) can be used.

(1) Solid Core Optical Fiber

This optical fiber has one solid core 52 having a refractive index higher than that of a clad 60 in the clad 60. “Solid” means “not hollow”. Note that the solid core can also be realized by forming an annular low refractive index region in the clad.

(2) Hole-Assisted Optical Fiber

This optical fiber has the solid core 52 and a plurality of holes 53 disposed on the outer periphery of the solid core 52 in the clad 60. The medium of the hole 53 is air, and the refractive index of the air is sufficiently smaller than that of quartz glass. Therefore, the hole-assisted optical fiber has a function of returning light leaking from the core 52 by bending or the like to the core 52 again, and has a characteristic of small bending loss.

(3) Hole-Structured Optical Fiber

This optical fiber has a hole group 53a of the plurality of holes 53 in the clad 60, and has a refractive index effectively lower than that of a host material (glass or the like). This structure is called a photonic crystal fiber. This structure can have a structure in which a high refractive index core having a changed refractive index is not present, and light can be confined by making a region 52a surrounded by the holes 53 an effective core region. As compared with the optical fiber having the solid core, the photonic crystal fiber can reduce the influence of absorption by an additive of the core and scattering loss, and can realize optical characteristics which cannot be realized by the solid optical fiber such as reduction of bending loss and control of nonlinear effect.

(4) Hollow Core Optical Fiber

The core region of this optical fiber is formed of air. Light can be confined in the core region by taking a photonic band gap structure by a plurality of holes or an anti-resonant structure by a glass thin wire in the clad region. The optical fiber has a small nonlinear effect and can supply a high output or high energy laser.

(5) Coupled Core Type Optical Fiber

In this optical fiber, a plurality of solid cores 52 having a high refractive index are disposed in close proximity to each other in the clad 60. The optical fiber guides light by light wave coupling between the solid cores 52.

Since the coupled core type optical fiber can disperse by the number of cores and send the dispersed light, there is an advantage that the power can be increased by that amount and efficient sterilization can be performed, or the deterioration of the fiber due to the ultraviolet rays can be relaxed and the service life can be prolonged.

In addition, the optical fiber having a cross-sectional structure as illustrated in FIG. 5 (6) to (10) can be used as the multi-core optical fiber (50-2, 53).

(6) Solid Core Type Multi-Core Optical Fiber

In this optical fiber, the plurality of solid cores 52 having the high refractive index are disposed apart from each other in the clad 60.

This optical fiber guides light in a state where the influence of the light wave coupling can be ignored by sufficiently reducing the light wave coupling between the solid cores 52.

Therefore, the solid core type multi-core optical fiber has an advantage that each core can be handled as an independent waveguide.

(7) Hole-Assisted Type Multi-Core Optical Fiber

This optical fiber has a structure in which a plurality of hole structures and core regions of the above (2) is disposed in the clad 60.

(8) Hole-Structured Type Multi-Core Optical Fiber

This optical fiber has a structure in which a plurality of hole structures of the above (3) is disposed in the clad 60.

(9) Hollow Core Type Multi-Core Optical Fiber

The optical fiber has a structure in which a plurality of the hole structures of the above (4) are disposed in a clad 60.

(10) Coupled Core Type Multi-Core Optical Fiber

The optical fiber has a structure in which a plurality of coupled core structures of the above (5) is disposed in the clad 60.

By using the optical fiber and the optical cable as described with reference to FIGS. 2 to 5 for the optical transmission line 50, the ultraviolet light can be transmitted to the respective irradiation parts 13, and it can be laid even in a fine place where a conventional robot or device cannot enter.

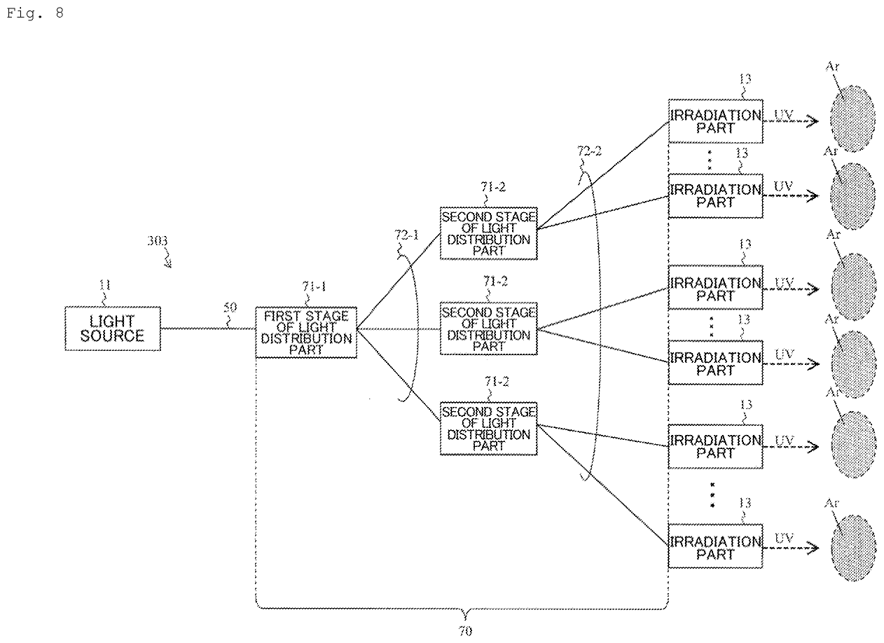

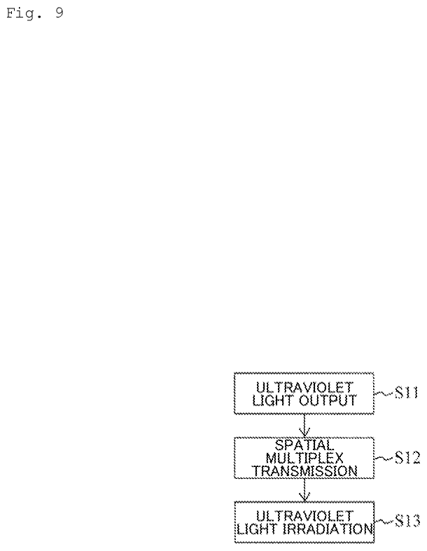

FIG. 6 is a diagram for describing the structure of the ultraviolet light source part 11. The ultraviolet light source part 11 has a light source 11a for outputting the ultraviolet light and an optical system 11b which couples the ultraviolet light with the core 52 (where, a region 52a surrounded by a hole group in the case of a structure shown in FIG. 3 and FIG. 8 and a hole 53c in the case of a structure shown in FIG. 4 and FIG. 9 are equivalent) of the optical fiber of the optical transmission line The ultraviolet light source part 11 has three structures as shown in FIG. 6 by the number of the light sources 11a and the number of the cores 52 of the optical fibers of the optical transmission line 50.

FIG. 6 (A) shows a structure in which the number of light sources 11a and the number of cores 52 of the optical fiber are the same. The light source 11a is a semiconductor light source such as an LD (Laser Diode) or an LED (Light Emitting Diode), a light source using nonlinear optics, or a lamp light source. The optical system 11b inputs the output light of each light source 11a to the core 52. This structure is simpler in design of the optical system and lower in cost than the structure shown in FIGS. 6 (B) and (C).

FIG. 6 (B) is a structure in which the number of light sources 11a is larger than the number of cores 52 of the optical fiber. The light source 11a is the same as the explanation in FIG. 6 (A). The optical system 11b inputs outputs of two or more light sources 11a to the single-core 52. In this structure, the power of the ultraviolet light to be transmitted can be increased by inputting the ultraviolet light from the plurality of light sources 11a to one core 52, and the object place Ar in a wide range can be sterilized or the like in a short time.

FIG. 6 (C) is a structure in which the number of light sources 11a is smaller than the number of cores 52 of the optical fiber. The light source 11a is the same as the explanation in FIG. 6 (A). The optical system 11b inputs the output light of the single light source 11a to the plurality of cores 52. In this structure, even if the power of the ultraviolet light transmittable by each core 52 is limited, the ultraviolet light of large power can be irradiated to the target place Ar by transmitting the power of the high-output light source 11a while dispersing it.

### Embodiment 2

FIG. 7 is a diagram for describing an ultraviolet light irradiation system 302 of the present embodiment. The ultraviolet light irradiation system 302 includes a plurality of irradiation parts 13, and further includes an optical distribution part 70 for distributing the ultraviolet light propagated by the optical transmission part 50 to the respective irradiation parts 13, as compared with the ultraviolet light irradiation system 301 described with reference to FIG. 1.

The optical distribution part 70 has an optical distributor 71 and an optical transmission part 72.

The optical distributor 71 distributes the ultraviolet light transmitted by the optical transmission line 50 to a plurality of optical transmission parts 72. For example, the light distribution part 71 is a fan-in fan-out device.

The optical transmission part 72 is an optical fiber having a structure of any one of the structures shown in FIG. 5 (1) to (10).

For example, when the optical transmission line 50 is an optical cable 50-1 described with reference to FIG. 2 or a multi-core optical fiber 50-2 described with reference to FIG. 3 and the optical transmission part 72 is a single-core optical fiber having a structure of any one of FIG. 5 (1) to (5), the optical distribution part 71 connects each optical fiber 51 in the optical cable 50-1 or each core 52 of the multi-core optical fiber 50-2 on the input side with each core of the single-core optical fiber on the output side one on one.

In addition, when the optical transmission line 50 is the optical cable 50-3 described with reference to FIG. 4 and the optical transmission part 72 is the multi-core optical fiber having any structure of FIG. 5 (6) to (10), the optical distribution part 71 connects each multi-core optical fiber 57 in the optical cable 50-3 on the input side and each multi-core optical fiber on the output side one on one.

That is, as the optical transmission line 50,
(X1) the optical cable 50-1 formed by bundling the single-core optical fibers having any structure of FIG. 5 (1) to (5),
(X2) the multi-core optical fiber 50-2 having any structure of FIG. 5 (6) to (10), or
(X3) the optical cable 50-3 formed by bundling the multi-core optical fibers 57 having any structure of FIG. 5 (6) to (10) can be used, and
as the respective transmission lines of the optical transmission line 72,
(Y1) the single-core optical fiber having any structure of FIG. 5 (1) to (5),
(Y2) the optical cable 50-1 formed by bundling the single-core optical fibers having any structure of FIG. 5 (1) to (5),
(Y3) the multi-core optical fiber 50-2 having any structure of FIG. 5 (6) to (10), or
(Y4) the optical cable 50-3 formed by bundling the multi-core optical fibers 57 having any structure of FIG. 5 (6) to (10) can be used.

The ultraviolet light irradiation system 302 can be constituted by any combination of any one of X1 to X3 and any one of Y1 to Y4.

### Embodiment 3

FIG. 8 is a diagram illustrating an ultraviolet light irradiation system 303 of the present embodiment. The ultraviolet light irradiation system 303 differs from the ultraviolet light irradiation system 302 described with reference to FIG. 7 in that the optical distributor 71 of the optical distribution part 70 is formed in multiple stages.

The optical distribution part 70 has the optical distributors (71-1, 71-2), and the optical transmission parts (72-1, 72-2). The optical distribution part 71-1 distributes the ultraviolet light transmitted through the optical transmission line 50 to a plurality of optical transmission parts 72-1. The optical distribution part 71-2 distributes the ultraviolet light transmitted by the optical transmission part 72-1 to a plurality of optical transmission parts 72-2. For example, the optical distribution parts (71-1, 71-2) are fan-in fan-out devices. The optical transmission parts (72-1, 72-2) are optical fibers having any structure of FIG. 5 (1) to (10).

For example, when the optical transmission line 50 is the optical cable 50-3 described with reference to FIG. 4, the optical transmission part 72-1 is the multi-core optical fiber having any structure of FIG. 5 (6) to (10), and the optical transmission part 72-2 is the single-core optical fiber having any structure of FIG. 5 (1) to (5),
the optical distribution part 71-1 connects each multi-core optical fiber 57 in the optical cable 50-3 on the input side and each multi-core optical fiber on the output side one on one, the optical distribution part 71-2 connects each core in the multi-core optical fiber on the input side and each core of the single-core optical fiber on the output side one on one.

Also in this embodiment, as the optical transmission line 50,
(X1) the optical cable 50-1 formed by bundling the single-core optical fibers having any structure of FIG. 5 (1) to (5),
(X2) the multi-core optical fiber 50-2 having any structure of FIG. 5 (6) to (10), or
(X3) the optical cable 50-3 formed by bundling the multi-core optical fibers 57 having any structure of FIG. 5 (6) to (10) can be used, and
as the respective transmission lines of the optical transmission line (72-1, 72-2),
(Y1) the single-core optical fiber having any structure of FIG. 5 (1) to (5),
(Y2) the optical cable 50-1 formed by bundling the single-core optical fibers having any structure of FIG. 5 (1) to (5),
(Y3) the multi-core optical fiber 50-2 having any structure of FIG. 5 (6) to (10), or
(Y4) the optical cable 50-3 formed by bundling the multi-core optical fibers 57 having any structure of FIG. 5 (6) to (10) can be used.

The ultraviolet light irradiation system 303 can be constituted by any combination of any one of X1 to X3 and any one of Y1 to Y4. In addition, in FIG. 8, the optical distributor 71 of the light distribution part 70 has been described in two stages, but the optical distribution part 70 of the ultraviolet light irradiation system 303 may be constituted by three or more optical distributors 71.

(Ultraviolet Light Irradiation Method)

FIG. 9 is a flowchart for describing an ultraviolet light irradiation method of the ultraviolet light irradiation system 301 of FIG. 1. The ultraviolet light irradiation method is characterized in that a step S12 of propagating the ultraviolet light by the spatial multiplex transmission scheme is performed when irradiating a desired place from an irradiation part 13 in a step S13 with the ultraviolet light outputted from the ultraviolet light source part 11 in a step S11.

In the step S12, the spatial multiplex transmission scheme is realized by the optical cable in which a plurality of single-core optical fibers is bundled, one multi-core optical fiber, or the optical cable in which a plurality of multi-core optical fibers is bundled.

In the step S13, when there is a plurality of desired places, the ultraviolet light propagated by the spatial multiplex transmission scheme may be distributed to the respective desired places.

### REFERENCE SIGNS LIST

11 Ultraviolet light source part

11a Light source

11b Optical system

11c Polarization combining part

13 Irradiation part

50 Optical transmission part

50-1 Optical cable

50-2 Multi-core optical fiber

50-3 Optical cable

51 Single-core optical fiber

52 Core

52a Region

53 Hole

53a Hole group

53c Hole

57 Multi-core optical fiber

60 Clad

301 to 303 Ultraviolet light irradiation system

1. An ultraviolet light irradiation system comprising:
an ultraviolet light source unit configured to output ultraviolet light;
an optical transmission part configured to propagate the ultraviolet light by a spatial multiplex transmission scheme; and
an irradiation part configured to irradiate a desired place with the ultraviolet light.

2. The ultraviolet light irradiation system according to claim 1, wherein
the optical transmission part is an optical cable formed by bundling a plurality of single-core optical fibers.

3. The ultraviolet light irradiation system according to claim 2, wherein
the single-core optical fiber is any one of a solid core optical fiber, a hole-assisted optical fiber, a hole-structured optical fiber, a hollow core optical fiber, and a coupled core type optical fiber.

4. The ultraviolet light irradiation system according to claim 1, wherein
the optical transmission part is one multi-core optical fiber or an optical cable formed by bundling a plurality of multi-core optical fibers.

5. The ultraviolet light irradiation system according to claim 4, wherein
the multi-core optical fiber is any one of a solid core type multi-core optical fiber, a hole- assisted type multi-core optical fiber, a hole-structured type multi-core optical fiber, a hollow core type multi-core optical fiber, and a coupled core type multi-core optical fiber.

6. The ultraviolet light irradiation system according to claim 1, wherein
the irradiation part is multiple, and
the ultraviolet light irradiation system further comprises:
an optical distribution part configured to distribute the ultraviolet light propagated by the optical transmission part to each irradiation part.

7. The ultraviolet light irradiation system according to 6, wherein the irradiation part and the optical distribution part are connected
by any one of the solid core optical fiber, the hole-assisted optical fiber, the hole-structured optical fiber, the hollow core optical fiber, the coupled core type optical fiber, the solid core type multi-core optical fiber, the hole-assisted type multi-core optical fiber, the hole-structured type multi-core optical fiber, the hollow core type multi-core optical fiber, and the coupled core type multi-core optical fiber.

8. An ultraviolet light irradiation method, wherein
when the ultraviolet light outputted from the ultraviolet light source part is irradiated from the irradiation part to the desired place,
the ultraviolet light is propagated by the spatial multiplex transmission scheme.
